(19) United States
(12) Patent Application Publication
(10) Pub. No.: US 2020/0115296 A1
(43) Pub. Date: Apr. 16, 2020

(54) METHODS OF PRODUCING LINEAR ALPHA OLEFINS

(71) Applicant: SABIC GLOBAL TECHNOLOGIES B.V., BERGEN OP ZOOM (NL)

(72) Inventors: Dafer Mubarak Al-Shahrani, Riyadh (SA); Shahid Azam, Riyadh (SA); Hans-Jörg Zander, Munich (DE); Wolfgang Muller, Munich (DE); Anina Wohl, Munich (DE); Andreas Meiswinkel, Munich (DE); Ralf Noack, Munich (DE); Tobias Meier, Munich (DE); Harald Schmaderer, Munich (DE)

(21) Appl. No.: 16/471,209

(22) PCT Filed: Dec. 19, 2017
(86) PCT No.: PCT/IB2017/058157

§ 371 Date: Jun. 19, 2019

Related U.S. Application Data
(60) Provisional application No. 62/438,123, filed on Dec. 22, 2016.

(51) Int. Cl. C07C 2/30; B01J 8/22; C07C 11/107

(52) U.S. Cl. CPC C07C 2/30; B01J 8/22; B01J 2208/00017; B01J 2208/00938; C07C 11/107; B01J 2208/00539

(57) ABSTRACT

A method of producing linear alpha olefins includes: introducing a catalyst stream and a first solvent stream to a reactor, wherein the reactor comprises a distributor; introducing a second solvent stream above the distributor, wherein the catalyst stream, the first solvent stream, and the second solvent stream form a reaction solution; introducing a feed stream to the reactor; passing the feed stream through the distributor; and passing the feed stream through the reaction solution, producing an oligomerization reaction forming the linear alpha olefins.

### BACKGROUND

Linear alpha olefins are olefins or alkenes with a chemical formula CₓH₂ₓ, distinguished from other mono-olefins with a similar molecular formula by linearity of the hydrocarbon chain and the position of the double bond at the primary or alpha position. There are a wide range of industrially significant applications for linear alpha olefins. For example, the lower carbon numbers, 1-butene, 1-hexene and 1-octene can be used as co-monomer in the production of polyethylene.

Linear alpha olefins can often be produced via the oligomerization of ethylene. This process presents many engineering challenges. For example, by-products such as dissolved polymer can form within the oligomerization reactor. As a result, gas distributors within the reactor can become clogged and fouled with deposits of the dissolved polymer, thus reducing the efficiency of the reactor and increasing maintenance requirements. For example, excessive hot flushing and mechanical cleaning of the reactor may be required to remove the polymer deposits.

Thus, there is a need for a treatment method that can produce a linear alpha olefin stream, reduce by-product formation within the reactor, and prevent polymer fouling of gas distributors, piping and other equipment.

### SUMMARY

Disclosed, in various embodiments, are methods of producing linear alpha olefins.

A method of producing linear alpha olefins, comprises: introducing a catalyst stream and a first solvent stream to a reactor, wherein the reactor comprises a distributor; introducing a second solvent stream above the distributor, wherein the catalyst stream, the first solvent stream, and the second solvent stream form a reaction solution; introducing a feed stream to the reactor; passing the feed stream through the distributor; and passing the feed stream through the reaction solution, producing an oligomerization reaction forming the linear alpha olefins.

A method of producing linear alpha olefins, comprises: introducing a catalyst stream and a first solvent stream comprising an alkane, an aromatic hydrocarbon, an olefin, or a combination comprising at least one of the foregoing to a reactor, wherein the reactor comprises a distributor; introducing a second solvent stream directly above and adjacent to the distributor, wherein the catalyst stream, the first solvent stream, and the second solvent stream form a reaction solution; introducing a feed stream comprising ethylene to the reactor; passing the feed stream through the distributor; and passing the feed stream through the reaction solution, producing an oligomerization reaction; wherein introduction of the second solvent stream dislodges polymer deposits located in and around the distributor and wherein the reactor, the distributor, reactor piping, downstream equipment, or a combination comprising at least one of the foregoing are relatively free of polymer and polymer deposits.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is a schematic diagram representing a reactor for producing linear alpha olefins.

### DETAILED DESCRIPTION

The method disclosed herein can produce a linear alpha olefin stream, reduce by- product formation within a reactor, and prevent polymer fouling of gas distributors, piping and other equipment. For example, the present method can produce a linear alpha olefin stream comprising greater than or equal to 91% hexene. The method can suppress and minimize the catalyst activity within an oligomerization reactor that leads to polymer formation. The method can also dislodge deposited polymer particles from gas distributors, piping and other equipment within the reactor. Accordingly, the method can further reduce overall capital costs and reduce the frequency and/or need for reactor maintenance.

The method disclosed herein can include introducing both a first solvent stream and a second solvent stream into an oligomerization bubble column reactor. The second solvent stream can be introduced directly above and adjacent to a gas distributor within the reactor. Accordingly, the second solvent stream can dislodge dissolved polymer deposits that are located in and around the distributor. The introduction of the second solvent stream can also dilute a reaction solution within the reactor and lower the catalyst concentration. This additional dilution can cause catalytic polymerization within the reactor to be suppressed. Accordingly, the formation of polymer deposits within the reactor, fouling, and associated maintenance requirements can be greatly reduced. Recycling of a product stream or a product fraction can also be possible with the method disclosed herein.

The method can include introducing a catalyst stream to a reactor. The catalyst stream can be gaseous and/or liquid. The catalyst stream can comprise an oligomerization catalyst. The catalyst can comprise a chromium source, a modifier, a ligand (e.g., a heteroatomic multidendate ligand), an organoaluminum compound (i.e., an activator), or a combination comprising at least one of the foregoing. A catalyst modifier is not required, but is also preferably present. For example, the catalyst can comprise a chromium compound and a ligand of the general structure (A) R₁R₂P—N(R₃)—P(R₄)—N(R₅)—H or (B) R₁R₂P—N(R₃)—P(R₄)—N(R₅)—PR₆R₇, wherein R₁-R₇ are independently selected from halogen, amino, trimethylsilyl, C₁-C₁₀-alkyl, C₆-C₂₀ aryl or any cyclic derivatives of (A) and (B), wherein at least one of the P or N atoms of the PNPN-unit or PNPNP-unit is a member of a ring system, the ring system being formed from one or more constituent compounds of structures (A) or (B) by substitution. The chromium compound can be an organic or inorganic salts, coordination complex, or organometallic complex of Cr(II) or Cr(III). In some embodiments the chromium compound is CrCl₃(tetrahydrofuran)₃, Cr(III)acetylacetonate, Cr(III)octanoate, chromium hexacarbonyl, Cr(III)-₂-ethylhexanoate, benzene(tricarbonyl)-chromium, or Cr(III)chloride. A combination of different chromium compounds can be used.

The heteroatomic multidentate ligand includes two or more heteroatoms (P, N, O, S, As, Sb, Bi, O, S, or Se) that can be the same or different, wherein the two or more heteroatoms are linked via a linking group. The linking group is a C₁₋₆ hydrocarbylene group or one of the foregoing heteroatoms. Any of the heteroatoms in the ligand can be substituted to satisfy the valence thereof, with a hydrogen, halogen, C₁₋₁₈ hydrocarbyl group, C₁₋₁₀ hydrocarbylene group linked to the same or different heteroatoms to form a heterocyclic structure, amino group of the formula NR^{a}R^{b} wherein each of R^{a} and R^{b} is independently hydrogen or a C₁₋₁₈ hydrocarbyl group, a silyl group of the formula SiR^{a}R^{b}R^{c} wherein each of R^{a}, R^{b}, and R^{c} is independently hydrogen or a C₁₋₁₈ hydrocarbyl group, or a combination comprising at least one of the foregoing substituents. The heteroatoms of the multidentate ligand are preferably a combination comprising phosphorus with nitrogen and sulfur or a combination comprising phosphorous and nitrogen, linked by at least one additional phosphorus or nitrogen heteroatom. In certain embodiments, the ligand can have the backbone PNP, PNPN, NPN, NPNP, NPNPN, PNNP, or cyclic derivatives containing these backbones wherein one or more of the heteroatoms is linked by a C₁₋₁₀ hydrocarbylene to provide a heterocyclic group. A combination of different ligands can be used.

In some embodiments, the ligand has the backbone PNPNH, which as used herein has the general structure R¹R²P—N(R³)—P(R⁴)—N(R⁵)—H wherein each of R¹, R², R³, R⁴, and R⁵ is independently a hydrogen, halogen, C₁₋₁₈ hydrocarbyl group, amino group of the formula NR^{a}R^{b} wherein each of R^{a} and R^{b} is independently hydrogen or a C₁₋₁₈ hydrocarbyl group, a silyl group of the formula SiR^{a}R^{b}R^{c} wherein each of R^{a}, R^{b}, and R^{c} is independently hydrogen or a C₁₋₁₈ hydrocarbyl group, or two of R¹, R², R³, R⁴, R⁵, R^{a}, or R^{b} taken together are a substituted or unsubstituted C₁₋₁₀ hydrocarbylene group linked to the same or different heteroatoms to form a heterocyclic structure. Exemplary ligands having a heterocyclic structure include the following

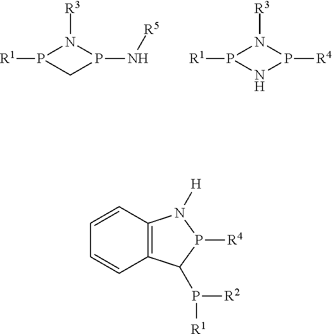

wherein R¹, R², R³, R⁴, R⁵ are as described above. In a specific embodiment, each R¹, R², R³, R⁴, R⁵ are independently hydrogen, substituted or unsubstituted C₁-C₈ alkyl, substituted or unsubstituted C₆-C₂₀ aryl, more preferably unsubstituted C₁-C₆ alkyl or unsubstituted C₆-C₁₀ aryl. A specific example of the ligand is (phenyl)₂PN(iso-propyl)P(phenyl)N(iso-propyl)H, commonly abbreviated Ph₂PN(i-Pr)P(Ph)NH(i-Pr).

Activators are known in the art, and are commonly aluminum compounds, for example a tri(C₁-C₆alkyl) aluminum such as triethyl aluminum, (C₁-C₆ alkyl) aluminum sesquichloride, di(C₁-C₆alkyl) aluminum chloride, or (C₁-C₆-alkyl) aluminum dichloride, or an aluminoxane such as methylaluminoxane (MAO). Each alkyl group can be the same or different, and in some embodiments is methyl, ethyl, isopropyl, or isobutyl. A combination of different activators can be used.

As is known in the art, the modifier can modify the activator, and serve as a chlorine source. Modifiers can include an ammonium or phosphonium salt of the type (H₄E)X, (H₃ER)X, (H₂ER₂)X, (HER₃)X, or (ER₄)X wherein E is N or P, X is Cl, Br, or I, and each R is independently a C₁-C₂₂ hydrocarbyl, preferably a substituted or unsubstituted C₁-C₁₆-alkyl, C₂-C₁₆-acyl, or substituted or unsubstituted C₆-C₂₀-aryl. In some embodiments the modifier is dodecyltrimethylammonium chloride or tetraphenylphosphonium chloride.

In some embodiments, the catalyst composition can be halogen-free. For example, the catalyst composition can be devoid of a halogenated compound, or no halogenated compound is intentionally added to the catalyst composition.

The catalyst composition is often pre-formed, for example by combining the components in a solvent before contacting with ethylene in an oligomerization process. Examples of solvents that can be used include toluene, benzene, ethylbenzene, cumenene, xylenes, mesitylene, C₄-C₁₅ paraffins, cyclohexane, C₄-C₁₂ olefins such as butene, hexene, heptene, octene, or ethers or multiethers such as diethylether, tetrahydrofuran, dioxane, di(C₁-C₈ alkyl)ethers. In some embodiments the solvent is an aromatic solvent such as toluene. The type of each component selected for use in the catalyst composition and relative amount of each component depend on the desired product and desired selectivity. In some embodiments, the concentration of the chromium compound is 0.01 to 100 millimole per liter (mmol/l), or 0.01 to 10 mmol/l, or 0.01 to 1 mmol/l, or 0.1 to 1.0 mmol/l; and the mole ratio of multidentate ligand:Cr compound:activator is 0.1:1:1 to 10:1:1,000, or 0.5:1:50 to 2:1:500, or 1:1:100 to 5:1:300. Suitable catalyst systems are described, for example, in EP2489431 B1; EP2106854 B1; and WO2004/056479.

The above-described catalyst composition can be contacted with a catalyst quenching medium to form a deactivated catalyst composition. The catalyst quenching medium comprises an alcohol having at least 6 carbon atoms, an organic amine, an amino alcohol, or a combination comprising at least one of the foregoing. In some embodiments, the catalyst quenching medium can be an organic amine, preferably a primary or secondary organic amine. For example, the organic amine can have the formula R⁶R⁷NH, wherein R⁶ and R⁷ are each independently hydrogen, a C₁₋₁₂ alkyl group, or a substituted or unsubstituted C₆₋₂₀ aryl group. In an embodiment, at least one of R⁶ or R⁷ is not a hydrogen. Examples of suitable organic amines can include tert-butyl amine, cyclopentylamine, tert-octylamine, n-heptylamine, ₂-heptylamine, hexylamine, 2-ethylhexylamine, dihexylamine, 1,6-diaminohexane, tributylamine, 1,8-diamineoctane, n-dodecylamine, 3-ethylheptylamine, and the like, or a combination comprising at least one of the foregoing. In some embodiments, the catalyst quenching medium is preferably an alcohol having at least 6 carbon atoms, for example a C₆₋₂₀ alkyl alcohol.

As used herein, the term “alcohol” includes monoalcohols, diols, and polyols. In some embodiments, the alcohol has a boiling point, or molecular weight, such that the alcohol will not form an azeotrope with the linear alpha olefin product. In some embodiments, the alcohol has a boiling point different from the olefin product in the reactor effluent stream. In some embodiments, the alcohol is a C₆₋₁₂ alkyl alcohol, for example 1-hexanol, 2-hexanol, 3-hexanol, 2-ethyl-l-hexanol, 1-heptanol, 2-heptanol, 3-heptanol, 4-heptanol, 2-methyl-3-heptanol, 1-octanol, 2-octanol, 3-octanol, 4-octanol, 7-methyl-2-decanol, 1-decanol, 2-decanol, 3-decanol, 4-decanol, 5-decanol, 2-ethyl-l-decanol, and combinations comprising at least one of the foregoing. In some embodiments, the catalyst quenching medium comprises 1-decanol.

A first solvent stream can also be introduced to the reactor. The first solvent stream can be gaseous and/or liquid. The first solvent stream can comprise an alkane, an aromatic hydrocarbon, an olefin, or a combination comprising at least one of the foregoing. For example, the first solvent stream can comprise aromatic hydrocarbons, straight chain aliphatic hydrocarbons, cyclic aliphatic hydrocarbons, ethers, toluene, benzene, ethylbenzene, cumene, xylenes, mesitylene, hexane, octane, cyclohexane, methylcyclohexane, diethylether, tetrahydrofurane, or a combination comprising at least one of the foregoing. For example, the first solvent stream can comprise toluene. The first solvent stream can be introduced to a top portion of the reactor. The catalyst stream and the first solvent stream can form a reaction solution, for example, a homogenous solution of catalyst and solvent within the reactor. Before addition of the second solvent stream, the concentration level of catalyst in the homogenous solution within the reactor can be 0.1% to 10% in the reaction zone.

The reactor can be a bubble column reactor, for example, for the oligomerization of ethylene. A temperature within the bubble column rector can be 30° C. to 120° C., for example, 35° C. to 100° C., for example, 40° C. to 80° C., for example, 30° C. to 90° C., for example, 45° C. to 60° C. A pressure within the bubble column reactor can be 1,000 kiloPascals to 5,000 kiloPascals, for example, 1,500 kiloPascals to 4,500 kiloPascals, for example, 2,000 kiloPascals to 4,000 kiloPascals.

The reactor can comprise a distributor. For example, the distributor can be a dynamic sparger plate and/or a gas bubbler. The distributor can be compressing or non-compressing. The distributor can comprise a single-phase jet nozzle, two-phase jet nozzle, ejector jet nozzle, momentum-transfer tube, venturi nozzle, or a combination comprising at least one of the foregoing. The distributor can be located in a bottom portion of the reactor. The reactor can comprise a single distributor or multiple distributors. The distributor(s) can be positioned in different arrangements. For example, the distributor(s) can be side mounted, dip-leg mounted, flanged-side mounted, cross mounted, or a combination comprising at least one of the foregoing.

A second solvent stream can be introduced to the reactor. The second solvent stream can be gaseous and/or liquid. The composition of the second solvent stream can be the same as, or different from, the composition first of the solvent stream. For example, the second solvent stream can comprise aromatic hydrocarbons, straight chain aliphatic hydrocarbons, cyclic aliphatic hydrocarbons, ethers, toluene, benzene, ethylbenzene, cumene, xylenes, mesitylene, hexane, octane, cyclohexane, methylcyclohexane, diethylether, tetrahydrofurane, or a combination comprising at least one of the foregoing. For example, the second solvent stream can comprise toluene.

The second solvent stream can be introduced to a bottom portion of the reactor. For example, the second solvent stream can be introduced directly above and adjacent to the distributor. Accordingly, the solvent from the second solvent stream can dislodge polymer particles deposited in and around the distributor. The solvent from the second solvent stream can combine with the reaction solution within the reactor. For example, the solvent from the second solvent stream can dilute the reaction solution. This additional dilution can lower the concentration of catalyst within the reaction solution and cause catalytic polymerization within the reactor to be suppressed. Accordingly, the formation of polymer deposits within the reactor, fouling, and associated maintenance requirements can be greatly reduced. For example, the reactor, the distributor, reactor piping, downstream equipment, or a combination comprising at least one of the foregoing can be relatively free of polymer and polymer deposits. Reactor distributor performance improvement is an indication of a reduction in polymer fouling. It was surprising to find an increase in reactor distributor performance with the method disclosed herein, which is an indicator of reduced polymer fouling (i.e., reduced polymer and polymer deposits in the reactor, the distributor, reactor piping, and/or downstream equipment).

After the second solvent stream has been introduced to the reactor, a feed stream can be introduced to the reactor. The feed stream can be gaseous and/or liquid. For example, the feed stream can comprise a mixture of hydrocarbons, for example, alkenes, for example, linear alpha olefins. The feed stream can comprise ethylene, ethane, methane, propane, ethylene, or a combination comprising at least one of the foregoing. The feed stream can comprise gaseous ethylene. The feed stream can comprise other hydrocarbons not listed as well as inert materials such as nitrogen.

The feed stream can be introduced to a bottom portion of the reactor. The feed stream can be passed though the distributor. The distributor can distribute the feed stream within the reactor. For example, the gaseous feed stream rise up through the reaction mixture and dissolve in the reaction mixture. Accordingly, an oligomerization reaction can occur within the reactor, for example, an ethylene oligomerization reaction.

1-Hexene is commonly manufactured by two general routes: (i) full-range processes via the oligomerization of ethylene and (ii) on-purpose technology. A minor route to 1-hexene, used commercially on smaller scales, is the dehydration of hexanol. Prior to the 1970s, 1-hexene was also manufactured by the thermal cracking of waxes. Linear internal hexenes were manufactured by chlorination/dehydrochlorination of linear paraffins.

“Ethylene oligomerization” combines ethylene molecules to produce linear alpha-olefins of various chain lengths with an even number of carbon atoms. This approach results in a distribution of alpha-olefins. Oligomerization of ethylene can produce 1-hexene. Fischer-Tropsch synthesis to make fuels from synthesis gas derived from coal can recover 1-hexene from the aforementioned fuel streams, where the initial 1-hexene concentration cut can be 60% in a narrow distillation, with the remainder being vinylidenes, linear and branched internal olefins, linear and branched paraffins, alcohols, aldehydes, carboxylic acids, and aromatic compounds. The trimerization of ethylene by homogeneous catalysts has been demonstrated.

The oligomerization reaction within the reactor can produce reaction products, for example, in the form of a gaseous and/or liquid effluent phase. The effluent phase can rise up within the reactor to a top portion of the reactor. The reaction products can be withdrawn from a top portion of the reactor via an effluent stream. The effluent stream can also be gaseous and/or liquid. The effluent stream can comprise hydrocarbons, such as linear alpha olefins, solvent and unreacted catalyst particles. For example, the effluent stream can comprise methane, ethylene, ethane, 1-butene, 1-hexene, toluene, 1-octane, 1-decene, 1-dodecene, catalyst particles, or a combination comprising at least one of the foregoing. For example, the effluent stream can comprise greater than or equal to 90% hexene, for example, greater than or equal to 91% hexene, for example, greater than or equal to 92% hexene. This can be true for a separated C6 fraction. The effluent stream can comprise 0 to 50 parts per million (ppm) unreacted catalyst particles. The effluent stream can also comprise by-products, such as dissolved polymer, for example, greater than or equal to 1 ppm dissolved polymer. The dissolved polymer can be a by-product of the oligomerization reaction that occurs within the reactor. The effluent stream can have a temperature of greater than or equal to 45° C. when withdrawn from the reactor.

The effluent stream can be transferred, via piping, from the reactor to subsequent downstream equipment, for example, a heat exchanger. The heat exchanger can utilize any desired cooling and/or heating means. For example, a heat exchanger fluid stream can be passed through the heat exchanger.

There are a wide range of applications for linear alpha olefins. The lower carbon numbers, 1-butene, 1-hexene and 1-octene can be used as comonomers in the production of polyethylene. High density polyethylene (HDPE) and linear low density polyethylene (LLDPE) can use approximately 2-4% and 8-10% of comonomers, respectively.

Another use of C₄-C₈ linear alpha olefins can be for production of linear aldehyde via oxo synthesis (hydroformylation) for later production of short-chain fatty acid, a carboxylic acid, by oxidation of an intermediate aldehyde, or linear alcohols for plasticizer application by hydrogenation of the aldehyde.

An application of 1-decene is in making polyalphaolefin synthetic lubricant base stock (PAO) and to make surfactants in a blend with higher linear alpha olefins. C₁₀-C₁₄ linear alpha olefins can be used in making surfactants for aqueous detergent formulations. These carbon numbers can be reacted with benzene to make linear alkyl benzene (LAB), which can be further sulfonated to linear alkyl benzene sulfonate (LABS), a popular relatively low cost surfactant for household and industrial detergent applications.

Although some C₁₄ alpha olefin can be sold into aqueous detergent applications, C₁₄ has other applications such as being converted into chloroparaffins. A recent application of C₁₄ is as on-land drilling fluid base stock, replacing diesel or kerosene in that application. Although C₁₄ is more expensive than middle distillates, it has a significant advantage environmentally, being much more biodegradable and in handling the material, being much less irritating to skin and less toxic.

C₁₆-C₁₈ linear olefins find their primary application as the hydrophobes in oil-soluble surfactants and as lubricating fluids themselves. C₁₆-C₁₈ alpha or internal olefins are used as synthetic drilling fluid base for high value, primarily off-shore synthetic drilling fluids. The preferred materials for the synthetic drilling fluid application are linear internal olefins, which are primarily made by isomerizing linear alpha-olefins to an internal position. The higher internal olefins appear to form a more lubricious layer at the metal surface and are recognized as a better lubricant. Another application for C₁₆-C₁₈ olefins is in paper sizing. Linear alpha olefins are, once again, isomerized into linear internal olefins and are then reacted with maleic anhydride to make an alkyl succinic anhydride (ASA), a popular paper sizing chemical.

C₂₀-C₃₀ linear alpha olefins production capacity can be 5-10% of the total production of a linear alpha olefin plant. These are used in a number of reactive and non-reactive applications, including as feedstocks to make heavy linear alkyl benzene (LAB) and low molecular weight polymers used to enhance properties of waxes.

The use of 1-hexene can be as a comonomer in production of polyethylene. High-density polyethylene (HDPE) and linear low-density polyethylene (LLDPE) use approximately 2-4% and 8-10% of comonomers, respectively.

Another use of 1-hexene is the production of the linear aldehyde heptanal via hydroformylation (oxo synthesis). Heptanal can be converted to the short-chain fatty acid heptanoic acid or the alcohol heptanol.

A more complete understanding of the components, processes, and apparatuses disclosed herein can be obtained by reference to the accompanying drawings. These figures (also referred to herein as “FIG.”) are merely schematic representations based on convenience and the ease of demonstrating the present disclosure, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

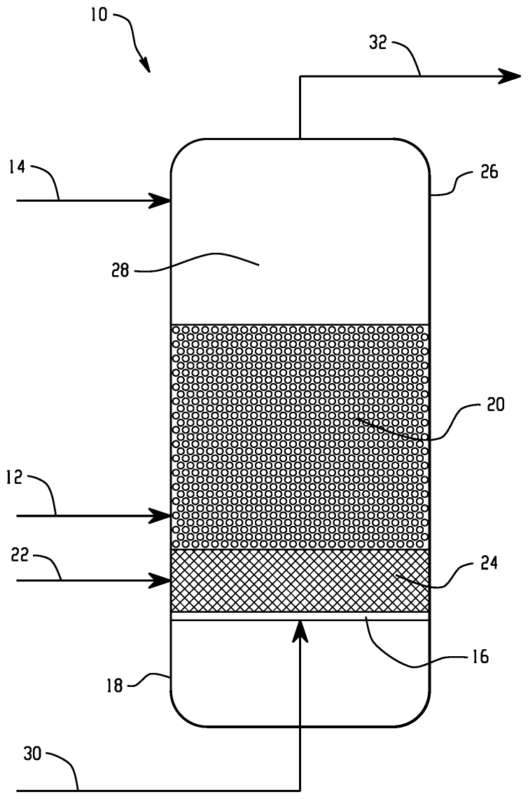

Referring now to FIG. 1, this schematic diagram represents a reactor 10 in a method of producing linear alpha olefins. The reactor 10 can be a bubble column reactor, for example, for the oligomerization of ethylene. The method can include introducing a catalyst stream 12 to the reactor 10. For example, the catalyst stream 12 can comprise an oligomerization catalyst. A first solvent stream 14 can also be introduced to the reactor 10. For example, the first solvent stream 14 can comprise toluene. The first solvent stream 14 can be introduced to a top portion 26 of the reactor 10. The catalyst stream 12 and the first solvent stream 14 can form a reaction solution 20, for example, a homogenous solution of catalyst and solvent.

The reactor 10 can comprise a distributor 16. The distributor 16 can be a gas sparger plate. The distributor 16 can be located in a bottom portion 18 of the reactor 10. A second solvent stream 22 can be introduced to the reactor 10. For example, the second solvent stream 22 can be introduced to a bottom portion 18 of the reactor 10 directly above and adjacent to the distributor 16. The solvent 24 from the second solvent stream 22 can combine with the reaction solution 20 within the reactor 10. For example, the solvent 24 from the second solvent stream 22 can dilute the reaction solution 20. The solvent 24 from the second solvent stream 22 can also dislodge polymer particles deposited in and around the distributor 16.

A feed stream 30 can be introduced to the reactor 10. For example, the feed stream 30 can comprise a mixture of hydrocarbons, for example, linear alpha olefins, for example, ethylene. The feed stream 30 can be introduced to a bottom portion 18 of the reactor 10. The feed stream 30 can be passed though the distributor 16. The distributor 16 can distribute the feed stream 30 within the reactor 10. For example, the feed stream 30 can pass through the reaction mixture 20 and dissolve in the reaction mixture 20. Accordingly, an oligomerization reaction can occur within the reactor 10.

This reaction can produce reaction products, for example, liner alpha olefins and polymer. The reaction products can form a gaseous effluent phase 28 that rises up within the reactor 10 and is withdrawn from the reactor 10. For example, the gaseous effluent phase 28 can comprise linear alpha olefins, solvent, catalyst and dissolved polymer. The effluent 28 can be withdrawn from a top portion 26 of the reactor 10 via an effluent stream 32.

The methods disclosed herein include(s) at least the following embodiments:

Aspect 1: A method of producing linear alpha olefins, comprising: introducing a catalyst stream and a first solvent stream to a reactor, wherein the reactor comprises a distributor; introducing a second solvent stream above the distributor, wherein the catalyst stream, the first solvent stream, and the second solvent stream form a reaction solution; introducing a feed stream to the reactor; passing the feed stream through the distributor; and passing the feed stream through the reaction solution, producing an oligomerization reaction forming the linear alpha olefins.

Aspect 2: The method of Aspect 1, wherein the catalyst stream comprises a chromium source, a modifier, a ligand, an organoaluminum compound, or a combination comprising at least one of the foregoing.

Aspect 3: The method of any of the preceding aspects, wherein the first solvent stream and/or the second solvent stream comprises an alkane, an aromatic hydrocarbon, an olefin, or a combination comprising at least one of the foregoing.

Aspect 4: The method of any of the preceding aspects, wherein the reactor is a bubble column reactor.

Aspect 5: The method of any of the preceding aspects, wherein the first solvent stream is introduced to a top portion of the reactor.

Aspect 6: The method of any of the preceding aspects, wherein an operating temperature within the reactor is 30° C. to 120° C., preferably, 35° C. to 100° C., more preferably 40° C. to 80° C.

Aspect 7: The method of any of the preceding aspects, wherein an operating pressure within the reactor is 2,000 kiloPascals to 4,000 kiloPascals.

Aspect 8: The method of any of the preceding aspects, wherein the distributor is located in a bottom portion of the reactor.

Aspect 9: The method of any of the preceding aspects, wherein the second solvent stream is introduced directly above and adjacent to the distributor.

Aspect 10: The method of any of the preceding aspects, wherein the distributor is a gas sparger plate and/or a gas bubbler.

Aspect 11: The method of any of the preceding aspects, wherein the feed stream comprises ethylene, ethane, methane, propane, or a combination comprising at least one of the foregoing.

Aspect 12: The method of any of the preceding aspects, further comprising forming reaction products.

Aspect 13: The method of Aspect 12, wherein the reaction products comprise hexene, solvent, catalyst, dissolved polymer, or a combination comprising at least one of the foregoing.

Aspect 14: The method of Aspect 13, wherein wherein a purity of a separated C6 fraction reaction product comprises greater than or equal to 91% hexene.

Aspect 15: The method of any of the preceding aspects, further comprising withdrawing the reaction products from the reactor via an effluent stream.

Aspect 16: The method of any of the preceding aspects, wherein the reactor, the distributor, reactor piping, downstream equipment, or a combination comprising at least one of the foregoing are relatively free of polymer and polymer deposits.

Aspect 17: The method of any of the proceeding aspects, wherein introduction of the second solvent stream to the reactor dilutes the reaction solution.

Aspect 18: The method of any of the preceding aspects, wherein introduction of the second solvent stream dislodges polymer deposits located in and around the distributor.

Aspect 19: The method of any of the preceding aspects, wherein the composition of the first solvent stream is different from the composition of the second solvent stream.

Aspect 20: A method of producing linear alpha olefins, comprising: introducing a catalyst stream and a first solvent stream comprising an alkane, an aromatic hydrocarbon, an olefin, or a combination comprising at least one of the foregoing to a reactor, wherein the reactor comprises a distributor; introducing a second solvent stream directly above and adjacent to the distributor, wherein the catalyst stream, the first solvent stream, and the second solvent stream form a reaction solution; introducing a feed stream comprising ethylene to the reactor; passing the feed stream through the distributor; and passing the feed stream through the reaction solution, producing an oligomerization reaction; wherein introduction of the second solvent stream dislodges polymer deposits located in and around the distributor and wherein the reactor, the distributor, reactor piping, downstream equipment, or a combination comprising at least one of the foregoing are relatively free of polymer and polymer deposits.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of producing linear alpha olefins, comprising:
introducing a catalyst stream and a first solvent stream to a reactor, wherein the reactor comprises a distributor;
introducing a second solvent stream above the distributor, wherein the catalyst stream, the first solvent stream, and the second solvent stream form a reaction solution;
introducing a feed stream to the reactor;
passing the feed stream through the distributor; and
passing the feed stream through the reaction solution, producing an oligomerization reaction forming the linear alpha olefins.

2. The method of claim 1, wherein the catalyst stream comprises a chromium source, a modifier, a ligand, an organoaluminum compound, or a combination comprising at least one of the foregoing.

3. The method of claim 1, wherein the first solvent stream and/or the second solvent stream comprises an alkane, an aromatic hydrocarbon, an olefin, or a combination comprising at least one of the foregoing.

4. The method of claim 1, wherein the reactor is a bubble column reactor.

5. The method of claim 1, wherein the first solvent stream is introduced to a top portion of the reactor.

6. The method of claim 1, wherein an operating temperature within the reactor is 30° C. to 120° C.

7. The method of claim 1, wherein an operating pressure within the reactor is 2,000 kiloPascals to 4,000 kiloPascals.

8. The method of claim 1, wherein the distributor is located in a bottom portion of the reactor.

9. The method of claim 1, wherein the second solvent stream is introduced directly above and adjacent to the distributor.

10. The method of claim 1, wherein the distributor is a gas sparger plate and/or a gas bubbler.

11. The method of claim 1, wherein the feed stream comprises ethylene, ethane, methane, propane, an inert gas.

12. The method of claim 1, further comprising forming reaction products.

13. The method of claim 12, wherein the reaction products comprise hexene, solvent, catalyst, dissolved polymer, or a combination comprising at least one of the foregoing.

14. The method of claim 13, wherein a purity of a separated C6 fraction reaction product comprises greater than or equal to 91% hexene.

15. The method of claim 1, further comprising withdrawing the reaction products from the reactor via an effluent stream.

16. The method of claim 1, wherein the reactor, the distributor, reactor piping, downstream equipment, or a combination comprising at least one of the foregoing are relatively free of polymer and polymer deposits.

17. The method of any of the proceeding claims claim 1, wherein introduction of the second solvent stream to the reactor dilutes the reaction solution.

18. The method of claim 1, wherein introduction of the second solvent stream dislodges polymer deposits located in and around the distributor.

19. The method of claim 1, wherein the composition of the first solvent stream is different from the composition of the second solvent stream.

20. A method of producing linear alpha olefins, comprising:
introducing a catalyst stream and a first solvent stream comprising an alkane, an aromatic hydrocarbon, an olefin, or a combination comprising at least one of the foregoing to a reactor, wherein the reactor comprises a distributor;
introducing a second solvent stream directly above and adjacent to the distributor, wherein the catalyst stream, the first solvent stream, and the second solvent stream form a reaction solution;
introducing a feed stream comprising ethylene to the reactor;
passing the feed stream through the distributor; and
passing the feed stream through the reaction solution, producing an oligomerization reaction;
wherein introduction of the second solvent stream dislodges polymer deposits located in and around the distributor and wherein the reactor, the distributor, reactor piping, downstream equipment, or a combination comprising at least one of the foregoing are relatively free of polymer and polymer deposits.
